(19) United States
(12) Patent Application Publication
(10) Pub. No.: US 2007/0015166 A1
(43) Pub. Date: Jan. 18, 2007

(54) Lateral flow methods and devices for detection of nucleic acid binding proteins

(76) Inventor: Thor W. Nilsen, Stirling, NJ (US)

Correspondence Address: Datascope Investment Corp., 14 Philips Parkway, Montvale, NJ, 07645, US

(21) Appl. No.: 11/181,260

(22) Filed: Jul. 14, 2005

(51) Int. Cl. C12Q 1/68

(52) U.S. Cl. 435/6

(57) ABSTRACT

Methods and devices are provided for detecting the presence or absence of nucleic acid binding proteins, such as NMP22, and other proteins, in bodily fluids.

### FIELD OF THE INVENTION

The present invention relates generally to lateral flow methods and devices for detection of nucleic acid binding proteins, such as NMP22, and other proteins, in bodily fluids.

### BACKGROUND OF THE INVENTION

Improving the performance of diagnostic assays is an ongoing challenge. Besides standard performance indicators such as sensitivity, specificity, and reproducibility, factors such as speed and related costs have become increasingly important. In this context, lateral flow assays have an acknowledged position and are well suited for rapid onsite testing. Most lateral flow tests reported to date relate to immunodiagnostics and are based on the specific interaction between antigens and antibodies.

Malignant conditions and cellular injury release cellular proteins which can be detected in readily available bodily fluids. Indeed the abnormal release of nuclear proteins, in particular proteins that bind nucleic acids, is a hallmark of cancer (see Abbas, Cell 84:655 (1996); Butler and Cerami, Nature 320:584 (1986); Galvan et al., Cancer Res. 42:1562 (1982); Hoon and Taback, Ann. N.Y. Acad. Sci. 1022:1 et seq. (2004); Keane et al., Cancer Res. 56:4791 (1996); Mak et al., Br. J. Surg. 91:790 (2004); Nagata, Cell 88:355 (1997); Simao et al., Braz. J. Med. Biol. Res. 32:403 (1999); Tanaka et al., Nat. Med. 2:317 (1996); Wyllie, Cancer Metastasis Rev. 11:95 (1992)). These proteins are expected to be present in greater quantities in individuals with malignant (both pre-cancerous, and cancerous) and cellular injury (heart attack, stroke, blunt force trauma, etc.) conditions. Blood, urine, saliva, sputum, exudates, tear drops, etc., are therefore expected to have a higher concentration of nucleic acid binding proteins than healthy individuals.

Various U.S. Patents to Matritech (e.g., U.S. Pat. Nos. 6,803,189, 6,740,494, 6,218,131, and 5,830,677) disclose immunoassays for the detection of various nucleic acid binding proteins, the presence of which serve as markers for various cancers, including bladder cancer, cervical cancer, colon cancer and breast cancer. One commercial embodiment, the NMP22 BladderChek® device, has been approved for the diagnosis of bladder cancer. The lateral flow device utilizes an immobilized anti-NMP22 antibody as the capture reagent to detect urinary NMP22, a marker for bladder cancer.

Such an assay could in theory be applied to other nucleic acid binding proteins found elevated in the body fluids of individuals with malignant and cellular injury conditions. Lateral flow assays based on antibody directly immobilized in the capture zone, however, suffer from various drawbacks, including denaturation, aggregation, precipitation, variability and nonspecific and suboptimal binding. Several lateral flow devices and assays have been described which utilize nucleic acids as capture reagents (see, e.g., Baeumner, Food Technology 58:51 (2004); Esch et al., Anal. Chem. 73:3162 (2001); Oku et al., J. Immunol. Methods 258:73 (2001); U.S. Pat. No. 6,037,127; U.S. Patent Publication No. 2004/0110167; U.S. Patent Publication No. 2003/0082571; International Patent Publication No. WO 95/24649); however, none of these references discloses the capture of nucleic acid binding proteins. As a result, there is an immediate need for lateral flow methods and devices and assays for detection of nucleic acid binding proteins, as well as other proteins, using immobilized nucleic acid molecules.

### SUMMARY OF THE INVENTION

Applicant has invented lateral flow methods and devices for detecting the presence or absence of a nucleic acid binding protein of interest in a fluid sample which utilize a nucleic acid molecule as the capture reagent. Applicant has also invented lateral flow methods and devices for detecting the presence or absence of a protein of interest in a fluid sample which utilize a capture reagent conjugated to a nucleic acid molecule.

Accordingly, one aspect of the present invention is directed to a lateral flow device for detecting the presence or absence of at least one nucleic acid binding protein of interest in a bodily fluid sample, said device comprising a test strip having a first and second end and comprising:

(a) a sample receiving zone at or adjacent said first end of said test strip for receiving an aliquot of a bodily fluid sample;

(b) a labeling zone in lateral flow contact with said sample receiving zone, said labeling zone comprising a porous material having at least a first detectable moiety reversibly bound thereto and coupled to a first binding partner which specifically binds to a nucleic acid binding protein of interest to form a detectable complex;

(c) a capture zone in lateral flow contact with said labeling zone, said capture zone comprising a microporous membrane, at least a portion of which contains one or more nucleic acid molecules immobilized thereto comprising a nucleic acid sequence which is bound by said nucleic acid binding protein of interest; and

(d) an absorbent zone positioned at or adjacent said second end of said test strip in lateral flow contact with capture zone,

wherein said detectable complex is captured by said nucleic acid molecule or molecules in said portion of said capture zone.

Another aspect of the present invention is directed to a method for detecting the presence or absence of at least one nucleic acid binding protein of interest in a bodily fluid sample, comprising:

(a) applying a bodily fluid sample to a sample receiving zone positioned at or adjacent a first end of a test strip of a lateral flow device, wherein said fluid sample laterally flows through said test strip sequentially from said sample receiving zone to a labeling zone to a capture zone to an absorbent zone positioned at or adjacent a second end of said test strip,

said labeling zone comprising a porous material having at least a first detectable moiety reversibly bound thereto and coupled to a first binding partner, wherein said first binding partner specifically binds to a nucleic acid binding protein of interest to form a detectable complex,

said capture zone comprising a microporous membrane, at least a portion of which contains one or more nucleic acid molecules immobilized thereto, wherein said nucleic acid molecule or molecules comprises a nucleic acid sequence which is bound by said nucleic acid binding protein of interest; and

(b) detecting the presence or absence of said nucleic acid binding protein in said portion of said capture zone.

Another aspect of the present invention is directed to a lateral flow device for detecting the presence or absence of at least one protein of interest in a bodily fluid sample, said device comprising a test strip having a first and second end and comprising:

(a) a sample receiving zone at or adjacent said first end of said test strip for receiving an aliquot of a bodily fluid sample;

(b) a labeling zone in lateral flow contact with said sample receiving zone, said labeling zone comprising a porous material having at least a first detectable moiety reversibly bound thereto and coupled to a first binding partner which specifically binds to a protein of interest to form a detectable complex;

(c) a capture zone in lateral flow contact with said labeling zone, said capture zone comprising a microporous membrane, at least a portion of which contains one or more nucleic acid molecules immobilized thereto, wherein said nucleic acid molecule or molecules are conjugated to a first capture reagent which specifically binds to said protein of interest; and

(d) an absorbent zone positioned at or adjacent said second end of said test strip in lateral flow contact with capture zone, wherein said detectable complex is captured by said first capture reagent in said portion of said capture zone.

Another aspect of the present invention is directed to a method for detecting the presence or absence of at least one protein of interest in a bodily fluid sample, comprising:

(a) applying a bodily fluid sample to a sample receiving zone positioned at or adjacent a first end of a test strip of a lateral flow device, wherein said fluid sample laterally flows through said test strip sequentially from said sample receiving zone to a labeling zone to a capture zone to an absorbent zone positioned at or adjacent a second end of said test strip,

said labeling zone comprising a porous material having at least a first detectable moiety reversibly bound thereto and coupled to a first binding partner, wherein said first binding partner specifically binds to a protein of interest to form a detectable complex,

said capture zone comprising a microporous membrane, at least a portion of which contains one or more nucleic acid molecules immobilized thereto, wherein said nucleic acid molecule or molecules are conjugated to a first capture reagent which specifically binds said protein of interest; and

(b) detecting the presence or absence of said protein of interest in said portion of said capture zone

In some embodiments of the present invention, the labeling zone of the lateral flow device further comprises a second control detectable moiety coupled to a second binding partner, and the capture zone further contains a control capture reagent which specifically binds to the second binding partner. In other embodiments, the capture zone of the lateral flow device further contains a control capture reagent which specifically binds to the first binding partner. Such control capture reagents are useful for verifying that the flow of fluid sample is as expected and that the detectable moieties have been successfully released from the labeling zone.

### BRIEF DESCRIPTION OF THE DRAWINGS

The accompanying drawings, which are incorporated in and form a part of the specification, illustrate non-limiting embodiments of the present invention, and together with the description serve to explain the principles of the invention. Wherever convenient, same or similar numbers or designations are used throughout the drawings to refer to the same or like elements.

FIG. 1 illustrates one embodiment of a test strip used in a lateral flow device for detecting the presence or absence of a nucleic acid binding protein of interest, wherein the sample receiving zone material, the labeling zone material, and the absorbent pad are each affixed to the capture zone membrane, which in turn is affixed to a semi-rigid or rigid support.

FIG. 2 illustrates another embodiment of a test strip used in a lateral flow device for detecting the presence or absence of a nucleic acid binding protein of interest, wherein the sample receiving zone material, the labeling zone material, the capture zone, and the absorbent pad are each affixed to a semi-rigid or rigid support.

FIGS. 3A and 3B illustrate a lateral flow assay for detecting the presence or absence of a nucleic acid binding protein of interest in a fluid sample using the lateral flow device illustrated in FIG. 1.

FIG. 4 illustrates one embodiment of a test strip used in a lateral flow device for detecting the presence or absence of a protein of interest, wherein the sample receiving zone material, the labeling zone material, and the absorbent pad are each affixed to the capture zone membrane, which in turn is affixed to a semi-rigid or rigid support.

FIGS. 5A and 5B illustrate a lateral flow assay for detecting the presence or absence of a protein of interest in a fluid sample using the lateral flow device illustrated in FIG. 4.

### DETAILED DESCRIPTION OF THE INVENTION

As used herein, the term “lateral flow” refers to the placement of a bodily fluid sample suspected of containing an analyte (e.g., nucleic acid binding protein) on a test strip comprising bibulous or non-bibulous material, wherein the analyte in the fluid sample flows laterally through the test strip by capillary action, coincidently reacting with various reagents in the strip.

As used herein, the term “test strip” refers to a chromatographic-like medium upon which an assay of this invention is preformed. Generally, the test strip contains in sequential order a “sample receiving zone” positioned at or adjacent the proximal (“first”) end for the application of the fluid sample, a “labeling zone” comprising detectable moieties coupled to a binding partner (e.g., antibody) that specifically binds to an analyte (e.g., nucleic acid binding protein) to form a detectable complex, a “capture zone” which contains an immobilized capture reagent (e.g., nucleic acid molecule) that captures and retains the detectable complex, and an absorbent pad positioned at or adjacent the distal (“second”) end to helps draw the fluid sample through the test strip.

As used herein, the term “bodily fluid sample” refers to any bodily fluid potentially containing an analyte. Such fluids include, but are not limited to, blood, plasma, serum, cerebrospinal fluid, urine, tears, saliva, semen, cervical secretions, vaginal secretions, breast milk, sweat, vomitus, synovial, pleural, peritoneal, pericardial, amniotic fluids, mucus, and cell lysate supernates, such as those obtained from buccal swabs, fine-needle aspirates, and tissue biopsies. The fluid can be obtained from any organism possessing such fluid, but is preferably obtained from a mammal, more preferably a human. The organism may be at risk for or suffering from cancer.

As used herein, the term “nucleic acid molecule” refers to a single stranded or double stranded RNA or DNA molecule composed of monomers (nucleotides) containing a sugar, phosphate and either a purine or pyrimidine. Such molecules can comprise nucleotides with backbone modifications, such as peptide nucleic acid (PNA), phosphorothioate DNA, phosphorodithioate DNA, phosphoramidate DNA, amide-linked DNA, MMI-linked DNA, 2′-O-methyl RNA, alpha-DNA and methylphosphonate DNA; nucleotides with sugar modifications, such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA; and nucleotides with base modifications, such as C-5 substituted pyrimidines (substituents including fluoro-, bromo-chloro-, iodo-, methyl-, ethyl-, vinyl-, formyl-, ethynyl-, propynyl-, alkynyl-, thiazoyl-, imidazolyl-, pyridyl-), 7-deazapurines with C-7 substituents including fluoro-, bromo-, chloro-, iodo-, methyl-, ethyl-, vinyl-, formyl-, alkynyl-, alkenyl-, thiazolyl-, imidazolyl-, pyridyl-), inosine and diaminopurine. Nucleic acid molecules include linear, branched, and dendritic nucleic acid molecules.

As used herein, the term “nucleic acid binding protein” refers to any peptide, polypeptide, or peptide-containing substance or complex that specifically interacts with a nucleic acid strand or strands. The nucleic acid binding protein may be a complex of two or more individual molecules, which may be the same (e.g., homodimer) or different (e.g., heterodimer). The nucleic acid protein may be sequence specific (e.g., transcription factors, restriction enzymes, sequence specific methylases, and DNA repair proteins), such that it binds to a specific sequence or family of specific sequences showing a high degree of sequence identity with each other (e.g., at least about 80% sequence identity) with generally at least 100-fold greater affinity than to unrelated sequences. Alternatively, the nucleic acid binding protein may be non-sequence specific (e.g., polymerases, nucleases, N-glycosylases, proteins of the telomerase complex, helicases, gyrases, topoisomerases, histones, splicing proteins, and any positively charged protein capable of binding nucleic acids), such that it binds to a plurality of unrelated DNA sequences with a dissociation constant that varies by less than 100-fold, usually less than tenfold, to the different sequences. Specific nucleic acid sequences may also be bound by more than one nonsequence specific nucleic acid binding protein.

As used herein, the term “binding partner” refers to a member of a pair of molecules and/or compositions capable of recognizing a specific structural aspect of another molecule or composition, wherein the binding partners interact with each other by means of a specific, noncovalent or covalent interaction. Examples of such binding partners and corresponding molecules or compositions include, but are not limited to, any of the class of immune-type binding pairs, such as antigen/antibody or hapten/anti-hapten systems; and also any of the class of nonimmune-type binding pairs, such as biotin/avidin, biotin/streptavidin, digoxigenin/anti-digoxigenin F(ab′)₂, folic acid/folate binding protein, complementary nucleic acid segments, protein A or G/immunoglobulins, lectin/carbohydrate, substrate/enzyme, inhibitor/enzyme, virus/cellular receptor; and binding pairs which form covalent bonds, such as sulfhydryl reactive groups including maleimides and haloacetyl derivatives, and amine reactive groups such as isotriocyanates, succinimidyl esters and sulfonyl halides.

As used herein, the term “detectable moiety” refers to any molecule or composition coupled to a binding partner capable of detection via optical means or energy emission, including, but not limited to, enzymes, particles, chemiluminescent moieties, bioluminescent moieties, fluorescent moieties, phosphorescent moieties, light emitting moieties, radionuclides, and electroactive compounds. Examples of suitable detectable moieties include colloidal gold and silver particles, colored latex and polystyrene particles, up-converting phosphor particles, rare-earth chelates (i.e., time resolved fluors), dye-encapsulated liposomes, labeled microspheres and microparticles, quantum dots, horseradish peroxidase, green fluorescent protein, fluorescein, ³⁵S, and acridinium esters.

As used herein, the term “detectable complex” refers to a complex between a detectable moiety and an analyte resulting from the interaction of detectable moiety-binding partner conjugate and the analyte.

As used herein, the term “specifically binds” refers to a binding reaction which is determinative of the presence of the analyte of interest in the presence of a heterogeneous population of molecules. Thus, the specified binding partner binds to a particular analyte and does not bind in a significant amount to other analytes present in the bodily fluid sample.

Generally, the devices and methods of the present invention employ lateral flow assay techniques as generally described in U.S. Pat. Nos. 5,770,460, 4,943,522; 4,861,711; 4,857,453; 4,855,240; 4,775,636; 4,703,017; 4,361,537; 4,235,601; 4,168,146; and 4,094,647.

The present invention provides, inter alia, lateral flow devices for detecting the presence or absence of a nucleic acid binding protein of interest in a bodily fluid sample. Such devices utilize a nucleic acid molecule bound by the nucleic acid binding protein of interest as the capture reagent.

Accordingly, one aspect of the present invention is directed to a lateral flow device for detecting the presence or absence of at least one nucleic acid binding protein of interest in a bodily fluid sample, said device comprising a test strip having a first and second end and comprising:

a sample receiving zone at or adjacent said first end of said test strip for receiving an aliquot of a bodily fluid sample;

a labeling zone in lateral flow contact with said sample receiving zone, said labeling zone comprising a porous material having at least a first detectable moiety reversibly bound thereto and coupled to a first binding partner which specifically binds to a nucleic acid binding protein of interest to form a detectable complex;

a capture zone in lateral flow contact with said labeling zone, said capture zone comprising a microporous membrane, at least a portion of which contains one or more nucleic acid molecules immobilized thereto comprising a nucleic acid sequence which is bound by said nucleic acid binding protein of interest;

an absorbent zone positioned at or adjacent said second end of said test strip in lateral flow contact with capture zone, wherein said detectable complex is captured by said nucleic acid molecule or molecules in said portion of said capture zone.

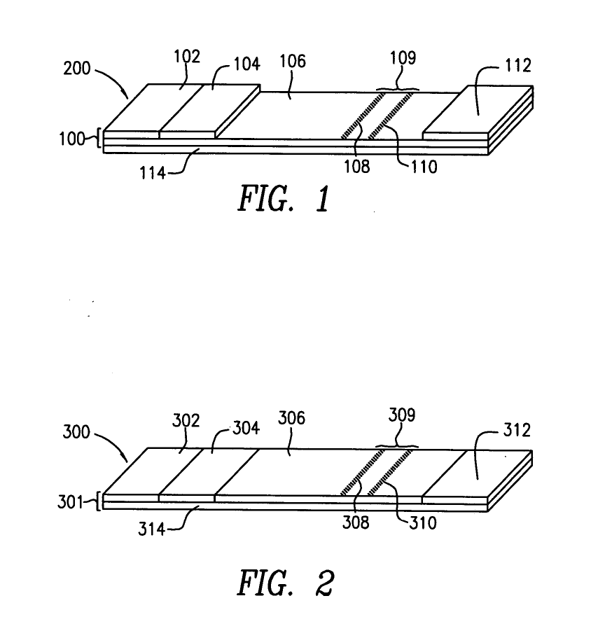

In this aspect of the present invention, one embodiment of a test strip 100 of a lateral flow device 200 of the present invention is shown in FIG. 1. In this embodiment, the capture zone material 106 extends the length of the test strip 100, and the sample receiving zone material 102 is affixed to the capture zone material 106. The sample receiving zone 102 serves to receive a bodily fluid sample which may contain the nucleic acid binding protein of interest and to begin the flow of the sample along the test strip 100. The sample receiving zone 102 is prepared from a natural or synthetic porous or macroporous material which is capable of conducting lateral flow of the fluid sample. A porous or macroporous material suitable for purposes of this invention generally has a pore size greater than 12 μm. Examples of porous materials include, but are not limited to, glass, cotton, cellulose, nitrocellulose, polyester, rayon, nylon, polyethersulfone, and polyethylene.

The labeling zone 104 comprises a material that is capable of conducting lateral flow and is in lateral flow contact with the sample receiving zone 102. In the embodiment shown in FIG. 1, the labeling zone material 104 is affixed to the capture zone material 106 on the same side as the sample receiving zone material. Materials suitable for the labeling zone material include, but are not limited to, porous or macroporous materials such as glass (e.g., borosilicate glass fiber), cotton, cellulose, nitrocellulose, polyester, polyethylene, rayon or nylon.

The labeling zone 104 also comprises at least a first detectable (“test”) moiety which is reversibly bound to the labeling zone material and is coupled to a binding partner. The binding partner is specific for the nucleic acid binding protein of interest. The labeling zone material must sufficiently retain the detectable moiety-binding partner conjugate in an anhydrous form prior to use of the lateral flow device, but must also release the conjugate upon contact with the fluid sample and allow lateral flow of the nucleic acid binding protein of interest both before and after binding to the conjugate. To minimize hindered flow caused by the inherent hydrophobic interactions between the labeling zone 104 and the detectable moiety-binding partner conjugate, the labeling zone material can be pretreated with a substance that maintains a small distance between the material and the conjugate, yet dissolves upon rehydration to allow the conjugate to flow to the capture zone 109. Examples of suitable substances include, but are not limited to, sucrose, various water soluble inert polymers, and surfactants.

Although the binding partner may be any molecule or composition that specifically binds a nucleic acid binding protein of interest, it will typically be an antibody. As used herein, the term “antibody” refers to a protein consisting of one or more polypeptides substantially encoded by immunoglobulin genes or fragments thereof. Antibodies to be used in the present invention can be produced by methods well known to those skilled in the art and include monoclonal antibodies, polyclonal antibodies, chimeric antibodies, humanized antibodies, single-chain antibodies, and fragments (e.g., F(ab′) and F(ab′)₂) thereof. Full-length protein may be used as the immunogen, or, alternatively, antigenic peptide fragments can be used. Antibodies specific for the protein can be identified by standard techniques, such as with ELISA, using immobilized marker protein. If desired, the antibodies can be isolated from the subject or culture media and further purified by standard techniques, such as protein A chromatography, to obtain an IgG fraction, or affinity chromatography.

The labeling zone 104 may also comprise a second detectable (“control”) moiety. The control moiety is carried through to the capture zone 109 along with the fluid flow. The control visible moiety is not coupled to a binding partner specific for the nucleic acid binding protein of interest. Rather, the control detectable moiety is coupled to a control binding partner that binds its specific binding partner that is immobilized in a separate “control” portion of the capture zone. The control detectable moiety is useful for verifying that the flow of fluid sample is as expected and that the detectable moieties have been successfully released from the labeling zone. The control detectable moiety may be the same as or different than the test detectable moiety. If different moieties are used, ease of reading the results is enhanced.

The capture zone material 106 comprises a microporous material which is capable of conducting lateral flow and is in lateral flow contact with the labeling zone material. Materials suitable for the capture zone membrane include, but are not limited to, microporous materials having a pore size from about 0.02 μm to 12 μm, such as cellulose, nitrocellulose, polyethersulfone, polyvinylidine fluoride, nylon, charge-modified nylon, and polytetrafluoroethylene. The capture zone 109 comprises a test capture region 108 containing one or more immobilized nucleic acid molecules comprising a nucleic acid sequence which is bound by the nucleic acid binding protein of interest. The nucleic acid sequence can comprise specific nucleotides for detection of sequence specific nucleic acid binding proteins or a plurality of unrelated nucleotides for detection of non-sequence specific nucleic acid binding proteins. The nucleic acid molecules act as capture moieties to capture any detectable complex formed as result of the detectable moiety-binding partner conjugate binding to the nucleic acid binding protein of interest in the labeling zone 104. The capture zone 109 can also contain an antibody that specifically binds the nucleic acid binding protein of interest to improve the efficiency of nucleic acid binding protein capture. Preferably, the capture antibody recognizes a different epitope than the labeling zone 104 antibody.

Methods for identifying proteins that bind specific nucleic acid sequences of interest, as well as identifying nucleic acid sequences that are bound by specific proteins of interest, are well known in the art and include, e.g., electrophoretic mobility shift assay (EMSA), supershift EMSA, Southwestern blotting, DNase I footprinting, affinity chromatography, ELISA, methylation interference assay, and UV crosslinking, as well as various bioinformatic approaches (e.g., Shanahan et al., Nucleic Acids Res. 32:4732 (2004)). The arrangement of the nucleic acid molecules in the test capture region 108 may be, for example, in the form of a dot, line, curve, band, cross, or combinations thereof. If the capture zone 109 also contains capture antibody, the antibody and nucleic acid molecules can be arranged as narrowly spaced separate lines in the test capture region 108 such that essentially a signal line is observed upon detection.

Methods of immobilizing nucleic acid molecules on the capture zone material 106 are well known in the art. For example, immobilization of the capture nucleic acid molecules directly on the capture zone material may be accomplished by using high salt to adsorb the nucleic acid molecules to its surface and baking at about 80° C. to permanently fix the adsorbed nucleic acid molecules. The nucleic acid molecules may also be fixed directly to the capture zone material by vacuum transfer in the presence of an equimolar concentration of sodium chloride and sodium citrate, or by the use of ultraviolet irradiation. The capture nucleic acid molecules may also be covalently linked to charge-modified nylon capture zone material. Alternatively, capture nucleic acid molecules may incorporate a reactive ligand (e.g., biotin) and may be immobilized indirectly on the capture zone material 106 as a result of the interaction between the ligand and an immobilized member of a binding pair (e.g., streptavidin).

The specific nucleic acid sequence recognized by the nucleic acid binding protein of interest can be presented in the test capture region 108 in single or multiple copies as a single or double stranded oligonucleotide, with the specific sequence generally ranging from about 3 nucleotides to about 25 nucleotides in length. The specific nucleic acid sequence may also be presented in single or multiple copies as part of a larger nucleic acid molecule or construct, for example, a restriction enzyme fragment, a plasmid, a branched nucleic acid molecule as described by Urdea et al. (e.g., U.S. Pat. No. 5,124,246), a dendrimeric nucleic acid molecule as described by Nilsen et al. (e.g., U.S. Pat. No. 5,175,270) or Luo et al (e.g., Li et al., Nat. Mat. 3:38 (2004)), a multimeric nucleic acid construct as described by Cantor et al. (e.g., U.S. Pat. No. 5,965,133), or an organized nucleic acid structure as described by Seeman et al. (e.g., U.S. Pat. Nos. 6,072,044 and 6,255,469). The use of larger nucleic acid molecules and constructs, particularly branched, dendrimeric, and multimeric species, increases the surface area available for binding to any detectable complex formed as result of the detectable moiety-binding partner conjugate binding to the nucleic acid binding protein of interest in the labeling zone 104.

If the labeling zone 104 contains a second control detectable moiety, the capture zone 109 may also contain a control capture reagent in a control capture region 110 that specifically binds the binding partner coupled to the second control detectable moiety. The arrangement of the control capture reagents in region 110 may be in the form of a dot, line, curve, band, cross, or combinations thereof. In one embodiment, as shown in FIG. 1, the immobilized control capture reagents are in a capture region 110 that is separate from the test capture region 108 that contains immobilized nucleic acid molecules bound by the nucleic acid binding protein of interest. Alternatively, the control capture reagents and the capture nucleic acid molecules are contained within the same region. In this embodiment, the first test and second control detectable moieties can comprise microparticles of different colors (e.g., blue and yellow), and the detection of a third color (e.g., green) in the capture zone indicates a positive result (i.e., the presence of the nucleic acid binding protein of interest). The control region 110 is useful in that appearance of a color in the control region 110 signals the time at which the test result can be read, even for a negative result (i.e., the absence of the nucleic acid binding protein of interest). Thus, when the expected color appears in the control region 110, the presence or absence of a color in the test region 108 can be noted.

Alternatively, the capture region 110 may contain a control capture reagent that specifically binds the binding partner coupled to the test detectable moiety. For example, if the binding partner coupled to the first test detectable moiety is a primary antibody, the capture reagent at capture region 110 can be a secondary antibody that recognizes and binds the primary antibody. When less than an excess of nucleic acid binding protein present in the fluid sample is bound by the test detectable moiety-binding partner conjugate in the labeling zone 104, some of the unbound detectable moiety-binding partner conjugate will be captured at the capture region 110. In addition to providing a control for the functionality of the test strip 100, the use of a control capture reagent in capture region 110 that specifically binds the binding partner coupled to the test detectable moiety may improve the (semi)-quantitation of the analyte when reading the test strip with a suitable reading device (as compared to the use of a control capture reagent that specifically binds a binding partner coupled to a second control detectable moiety) by providing a means for calculating a ratio of test line to control line both containing detectable moiety-binding partner conjugate.

The absorbent zone 112 is an absorbent material that is placed in lateral flow contact with the capture zone at the distal (“second”) end of the test strip. In the embodiment shown in FIG. 1, the absorbent zone material 112 is affixed to the capture zone material 106 on the same side sample receiving zone material and the labeling zone material. The absorbent pad material 112 helps to draw a bodily fluid sample from the sample receiving zone positioned at or adjacent the proximal (“first”) end of the test strip 100 to the distal (“second”) end of the test strip by capillary action. Examples of materials suitable for use as an absorbent pad include any absorbent material, including, but not limited to, nitrocellulose, cellulose esters, glass (e.g., borosilicate glass fiber), polyethersulfone, cotton, dehydrated polyacrylamide, silica gel, and polyethylene glycols. The rate of capillary flow can be controlled by choosing the appropriate absorbent zone material.

In the embodiment illustrated in FIG. 1, the capture zone material 106 is affixed to a rigid or semi-rigid support 114, which provides structural support to the test strip 100. The support can be made of any suitable rigid or semi-rigid material, such as poly(vinyl chloride), polypropylene, polyester and polystyrene. The membrane 106 may be affixed to the support 114 by any suitable adhesive means such as with a double-sided adhesive tape. Alternatively, the support 114 may be a pressure sensitive adhesive laminate, e.g., a polyester support having an acrylic pressure sensitive adhesive on one side that is optionally covered with a release liner prior to application to the membrane.

If desired, the lateral flow device 200 can be encased in a housing as described in, e.g., U.S. Pat. No. 5,451,504. Materials for use in the housing include, but are not limited to, transparent tape, plastic film, plastic, glass, metal and wood, with tape, plastic film and plastic preferred. The housing preferably has an opening to apply fluid sample to the sample receiving zone 102, and a window or windows above the capture zone 109 to observe results at capture regions 108 and 110.

In this aspect of the present invention, an alternative embodiment of a test strip 301 of a lateral flow device 300 of the present invention is shown in FIG. 2. In this embodiment, the sample receiving zone material 302, the labeling zone material 304, the capture zone material 306, and the absorbent material 312 are each affixed to a rigid or semi-rigid support 314. As shown, sample receiving zone material 302 overlaps with labeling zone material 304 to allow for lateral flow contact. Similarly, the labeling zone material 304 overlaps with the capture zone material 306, which overlaps with the absorbent zone material 312. While it is not required that materials 302, 304, 306, and 312 overlap as described, these materials must at least be in physical contact in the sequence shown in FIG. 2 such that the test sample can flow along the test strip 301 without interruption.

The present invention also provides methods for detecting the presence or absence of a nucleic acid binding protein of interest in a fluid sample.

Accordingly, another aspect of the present invention is directed to a method for detecting the presence or absence of at least one nucleic acid binding protein of interest in a bodily fluid sample, comprising:

(a) applying a bodily fluid sample to a sample receiving zone positioned at or adjacent a first end of a test strip of a lateral flow device, wherein said fluid sample laterally flows through said test strip sequentially from said sample receiving zone to a labeling zone to a capture zone to an absorbent zone positioned at or adjacent a second end of said test strip,

said labeling zone comprising a porous material having at least a first detectable moiety reversibly bound thereto and coupled to a first binding partner, wherein said first binding partner specifically binds to a nucleic acid binding protein of interest to form a detectable complex,

said capture zone comprising a microporous membrane, at least a portion of which contains one or more nucleic acid molecules immobilized thereto, wherein said nucleic acid molecule or molecules comprises a nucleic acid sequence which is bound by said nucleic acid binding protein of interest; and

(b) detecting the presence or absence of said nucleic acid binding protein in said portion of said capture zone.

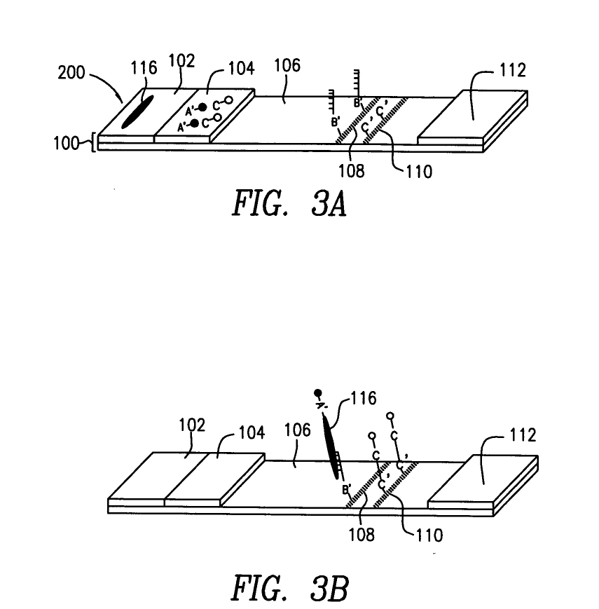

One embodiment of this aspect of the present invention using the lateral flow device shown in FIG. 1 is illustrated in FIGS. 3A and 3B. Beginning with FIG. 3A, the lateral flow device 200 comprises test strip 100 having sample receiving zone 102. A bodily fluid sample which may contain a nucleic acid binding protein of interest is applied to the sample receiving zone 102. Test strip 100 also contains a first detectable (“test”) moiety (black sphere) reversibly bound to the labeling zone material 104 and coupled to binding partner A′. Binding partner A′ is designed to specifically recognize and bind to any nucleic acid binding protein of interest 116 present in the fluid sample. Test strip 100 further comprises capture zone 108 containing capture nucleic acid molecule B′ immobilized on the capture zone material 106. Capture nucleic acid molecule B′ is designed to be recognized and bound by any nucleic acid binding protein of interest 116 bound by the detectable moiety-binding partner A′ conjugate.

As the fluid sample moves through the labeling zone 104, the test detectable moiety coupled to binding partner A′ is released form the labeling zone material and binds to nucleic acid binding protein of interest 116 to form a detectable complex. The bound detectable moiety thus flows along capture zone material 106 with the complex in the direction of the capture region 108 as shown in FIG. 3B. Upon reaching the capture region 108, nucleic acid binding protein of interest 116 is captured and immobilized in capture region 108 by capture nucleic acid molecule B′. Thus, if the nucleic acid binding protein of interest 116 is present in the fluid sample, the test detectable moieties will be collected and bound in the capture region 108, forming a detectable signal such as a colored line. Continued movement of the fluid sample draws excess reagents and unbound material (e.g., unbound test detectable moieties) past the capture region 108 to the absorbent zone 112.

The lateral flow methods embodied in FIGS. 3A and 3B can also incorporate the use of a second detectable (“control”) moiety to verify the functionality of the test strip 100. Thus, with reference to FIG. 3A, labeling zone 104 can further comprise a control detectable moiety (white sphere) reversibly bound to the labeling zone material and coupled to control binding partner C, and capture zone material 106 can comprise control capture reagent C′ immobilized in capture region 110. Control binding partner C and control capture reagent C′ are members of a binding pair that specifically recognize and bind to each other. During the method illustrated in FIGS. 3A and 3B, the control detectable moiety flows along capture zone material 106 with the fluid sample in the direction of the capture region 110. Upon reaching the capture zone 110, control binding partner C coupled to control detectable moiety are captured and immobilized in capture region 110 by control capture reagent C′, thus forming a detectable signal, e.g., a colored line. The control detectable moieties may be the same or a different color than the test detectable moieties. If different colors are used, ease of reading the results is enhanced. In an alternative embodiment, capture regions 108 and 110 overlap. In this embodiment, the first test and second control detectable moieties can comprise microparticles of different colors (e.g., blue and yellow), and the detection of a third color (in this case, green) in the capture zone indicates a positive result (i.e., the presence of the nucleic acid binding protein of interest).

Alternatively, the lateral flow methods embodied in FIGS. 3A and 3B can incorporate the use of a capture region 110 comprising a control capture reagent that specifically binds the binding partner coupled to the test detectable moiety. For example, if the binding partner coupled to the first test detectable moiety is a primary antibody, the capture reagent at capture region 110 can be a secondary antibody that recognizes and binds the primary antibody. When less than an excess of nucleic acid binding protein present in the fluid sample is bound by the test detectable moiety-binding partner conjugate in the labeling zone 104, some of the unbound detectable moiety-binding partner conjugate will be captured at the capture region 110, thereby providing a control for functionality of the test strip 100, as well as a means for calculating a ratio of test line to control line for the (semi)-quantitation of the nucleic acid binding protein of interest 116 in the fluid sample.

The present invention also provides lateral flow devices for detecting the presence or absence of a protein of interest in a bodily fluid sample. Such devices utilize a nucleic acid molecule conjugated to a capture reagent which specifically binds the protein of interest.

Accordingly, another aspect of the present invention is directed to a lateral flow device for detecting the presence or absence of at least one protein of interest in a bodily fluid sample, said device comprising a test strip having a first and second end and comprising:

(a) a sample receiving zone at or adjacent said first end of said test strip for receiving an aliquot of a bodily fluid sample;

(b) a labeling zone in lateral flow contact with said sample receiving zone, said labeling zone comprising a porous material having at least a first detectable moiety reversibly bound thereto and coupled to a first binding partner which specifically binds to a protein of interest to form a detectable complex;

(c) a capture zone in lateral flow contact with said labeling zone, said capture zone comprising a microporous membrane, at least a portion of which contains one or more nucleic acid molecules immobilized thereto, wherein said nucleic acid molecule or molecules are conjugated to a first capture reagent which specifically binds to said protein of interest; and

(d) an absorbent zone positioned at or adjacent said second end of said test strip in lateral flow contact with capture zone, wherein said detectable complex is captured by said first capture reagent in said portion of said capture zone.

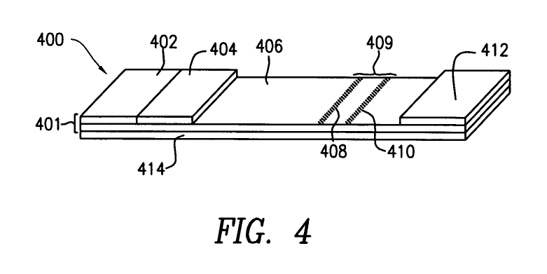

In this aspect of the present invention, an embodiment of a test strip 401 of a lateral flow device 400 is shown in FIG. 4. In this embodiment, the capture zone 409 comprises a test capture region 408 containing one or more immobilized nucleic acid molecules conjugated to a capture reagent that specifically binds a protein of interest. Although the capture reagent may be any molecule or composition that specifically binds a protein of interest, it will typically be an antibody. Preferably, the capture antibody recognizes a different epitope than the labeling zone 404 antibody.

When the protein of interest is a nucleic acid binding protein, the nucleic acid molecules used for capture reagent conjugation can comprise a nucleic acid sequence which is bound by the nucleic acid binding protein. As described above, the nucleic acid molecules can comprise specific nucleotide sequences for detection of sequence specific nucleic acid binding proteins or a plurality of unrelated nucleic acid sequences for detection of non-sequence specific nucleic acid binding proteins.

Methods for direct and indirect conjugation of antibodies to nucleic acid molecules are well known in the art and include biotin/streptavidin methodologies, chemical modification and crosslinking (see, e.g., Niemeyer, Biochem. Soc. Trans. 32:51 (2004); Niemeyer et al., Nucleic Acids. Res. 31:e90 (2003); Niemeyer, Trends Biotechnol. 20:395 (2002); Niemeyer et al., Bioconjug. Chem. 12:364 (2001); Niemeyer et al., Nucleic Acids. Res. 27:4553 (1999); Hendrickson et al., Nucleic Acids Res. 23:522 (1995); Joerger et al., Clin. Chem. 41:1371 (1995); Niemeyer et al., Nucleic Acids Res. 22:5530 (1994); Sano et al., Science 258:5079 (1992); U.S. Pat. No. 6,511,809; U.S. Pat. No. 5,985,548; U.S. Pat. No. 5,965,133; U.S. Pat. No. 5,665,539; U.S. Pat. No. 5,635,602). Any nucleic acid molecule can be used for conjugation, including oligonucleotides, restriction fragments, and plasmids. Preferably, the capture antibody is conjugated to a three-dimensional DNA molecule, such as a branched DNA molecule as described by Urdea et al. (e.g., U.S. Pat. No. 5,124,246), a dendrimeric DNA molecule as described by Nilsen et al. (e.g., U.S. Pat. No. 5,175,270) or Luo et al (e.g., Li et al., Nat. Mat. 3:38 (2004)), a multimeric DNA construct as described by Cantor et al. (e.g., U.S. Pat. No. 5,965,133), or an organized DNA structure as described by Seeman et al. (e.g., U.S. Pat. Nos. 6,072,044 and 6,255,469). The use of nucleic acid molecules for capture reagent conjugation reduces the denaturation and aggregation associated with antibody immobilization. In addition, the use of larger, three-dimensional nucleic acid molecules and constructs increases the available surface area for capture reagent immobilization, as well as decreasing the effective pore size of capture zone material 406, resulting in improved detectable complex capture.

The present invention also provides methods for detecting the presence or absence of a nucleic acid binding protein of interest in a fluid sample.

Accordingly, another aspect of the present invention is directed to a method for detecting the presence or absence of at least one protein of interest in a bodily fluid sample, comprising:

(a) applying a bodily fluid sample to a sample receiving zone positioned at or adjacent a first end of a test strip of a lateral flow device, wherein said fluid sample laterally flows through said test strip sequentially from said sample receiving zone to a labeling zone to a capture zone to an absorbent zone positioned at or adjacent a second end of said test strip,

said labeling zone comprising a porous material having at least a first detectable moiety reversibly bound thereto and coupled to a first binding partner, wherein said first binding partner specifically binds to a protein of interest to form a detectable complex,

said capture zone comprising a microporous membrane, at least a portion of which contains one or more nucleic acid molecules immobilized thereto, wherein said nucleic acid molecule or molecules are conjugated to a first capture reagent which specifically binds said protein of interest; and

(b) detecting the presence or absence of said protein of interest in said portion of said capture zone.

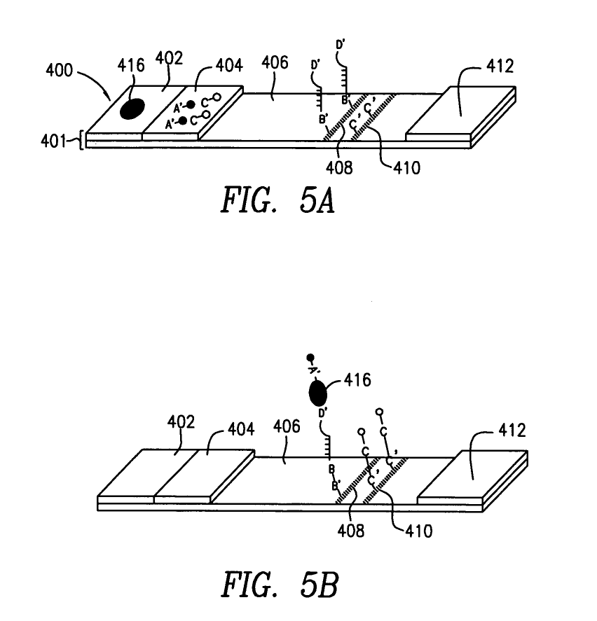

One embodiment of this aspect of the present invention using the lateral flow device shown in FIG. 4 is illustrated in FIGS. 5A and 5B. Beginning with FIG. 5A, the lateral flow device 400 comprises test strip 401 having sample receiving zone 402. A bodily fluid sample which may contain a binding protein of interest is applied to the sample receiving zone 402. Test strip 401 also contains a first detectable (“test”) moiety (black sphere) reversibly bound to the labeling zone material 404 and coupled to binding partner A′. Binding partner A′ is designed to specifically recognize and bind to any protein of interest 416 present in the fluid sample. Test strip 401 further comprises capture zone 408 containing a capture reagent D′ conjugated to a nucleic acid molecule B′ immobilized on the capture zone material 406. The capture reagent is designed to bind any protein of interest bound by the detectable moiety-binding partner A′ conjugate.

As the fluid sample moves through the labeling zone 404, the test detectable moiety coupled to binding partner A′ is released form the labeling zone material and binds to protein of interest 416 to form a detectable complex. The bound detectable moiety thus flows along capture zone material 406 with the complex in the direction of the capture region 408 as shown in FIG. 5B. Upon reaching the capture region 408, protein of interest 416 is captured and immobilized in capture region 408 by capture reagent D′. Thus, if protein of interest 416 is present in the fluid sample, the test detectable moieties will be collected and bound in the capture region 408, forming a detectable signal such as a colored line. Continued movement of the fluid sample draws excess reagents and unbound material (e.g., unbound test detectable moieties) past the capture region 408 to the absorbent zone 412. If protein of interest 416 is a nucleic acid binding protein, nucleic acid molecule B′ can comprise a nucleic acid sequence (either specific or nonspecific) known to be bound by nucleic acid binding protein of interest 416, thereby increasing the efficiency of detectable complex capture.

As described above, the lateral flow methods embodied in FIGS. 5A and 5B can also incorporate the use of a second detectable (“control”) moiety to verify the functionality of the test strip 401. Thus, with reference to FIG. 5A, labeling zone 404 can further comprise a control detectable moiety (white sphere) reversibly bound to the labeling zone material and coupled to control binding partner C, and capture zone material 406 can comprise control capture reagent C′ that specifically binds to control binding partner C immobilized in capture region 410. Alternatively, capture region 410 can comprise a control capture reagent that specifically binds the binding partner coupled to the test detectable moiety. For example, if the binding partner coupled to the first test detectable moiety is a primary antibody, the capture reagent at capture region 410 can be a secondary antibody that recognizes and binds the primary antibody.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1. A lateral flow device for detecting the presence or absence of at least one nucleic acid binding protein of interest in a bodily fluid sample, said device comprising a test strip having a first and second end and comprising:
(a) a sample receiving zone at or adjacent said first end of said test strip for receiving an aliquot of a bodily fluid sample;
(b) a labeling zone in lateral flow contact with said sample receiving zone, said labeling zone comprising a porous material having at least a first detectable moiety reversibly bound thereto and coupled to a first binding partner which specifically binds to a nucleic acid binding protein of interest to form a detectable complex;
(c) a capture zone in lateral flow contact with said labeling zone, said capture zone comprising a microporous membrane, at least a portion of which contains one or more nucleic acid molecules immobilized thereto comprising a nucleic acid sequence which is bound by said nucleic acid binding protein of interest; and
(d) an absorbent zone positioned at or adjacent said second end of said test strip in lateral flow contact with said capture zone, wherein said detectable complex is captured by said nucleic acid molecule or molecules in said portion of said capture zone.

2. The device of claim 1, wherein the capture zone extends the length of the test strip.

3. The device of claim 1, wherein the first binding partner is an antibody.

4. The device of claim 1, wherein the capture zone further contains in a portion thereof an antibody that specifically binds to the nucleic acid binding protein of interest.

5. The device of claim 1, wherein the nucleic acid molecule or molecules are in a form selected from the group consisting of oligonucleotide, restriction enzyme fragment, plasmid, branched nucleic acid molecule, dendrimeric nucleic acid molecule, multimeric nucleic acid construct and organized nucleic acid construct.

6. The device of claim 5, wherein the nucleic acid sequence comprises specific nucleotides which are bound by a sequence specific nucleic acid binding protein of interest.

7. The device of claim 5, wherein the nucleic acid sequence comprises unrelated nucleotides which are bound by a non-sequence specific nucleic acid binding protein of interest.

8. The device of claim 1, wherein the labeling zone further comprises a second control detectable moiety coupled to a second binding partner, and the capture zone further contains in a portion thereof a control capture reagent which specifically binds to said second binding partner.

9. The device of claim 1, wherein the capture zone further contains in a portion thereof a control capture reagent which specifically binds to the first binding partner.

10. A method for detecting the presence or absence of at least one nucleic acid binding protein of interest in a bodily fluid sample, comprising:
(a) applying a bodily fluid sample to a sample receiving zone positioned at or adjacent a first end of a test strip of a lateral flow device, wherein said fluid sample laterally flows through said test strip sequentially from said sample receiving zone to a labeling zone to a capture zone to an absorbent zone positioned at or adjacent a second end of said test strip,
said labeling zone comprising a porous material having at least a first detectable moiety reversibly bound thereto and coupled to a first binding partner, wherein said first binding partner specifically binds to a nucleic acid binding protein of interest to form a detectable complex,
said capture zone comprising a microporous membrane, at least a portion of which contains one or more nucleic acid molecules immobilized thereto, wherein said nucleic acid molecule or molecules comprises a nucleic acid sequence which is bound by said nucleic acid binding protein of interest; and
(b) detecting the presence or absence of said nucleic acid binding protein in said portion of said capture zone.

11. The method of claim 10, wherein the labeling zone further comprises a second control detectable moiety coupled to a second binding partner, and the capture zone further contains in a portion thereof a control capture reagent which specifically binds to said second binding partner.

12. The method of claim 10, wherein the capture zone further contains in a portion thereof a control capture reagent which specifically binds to the first binding partner.

13-24. (canceled)

25. The device of claim 8, wherein the portion of the capture zone that contains the control capture reagent is separate from the portion of the capture zone that contains the one or more nucleic acid molecules.

26. The device of claim 8, wherein the portion of the capture zone that contains the control capture reagent is the same as the portion of the capture zone that contains the one or more nucleic acid molecules.

27. The device of claim 8, wherein the first and second detectable moieties comprise microparticles of different colors.

28. The device of claim 1, wherein the capture zone is affixed to a rigid or semi-rigid support.

29. The device of claim 28, wherein the sample receiving zone, the labeling zone and the absorbent zone are also affixed to the rigid or semi-rigid support.

30. The device of claim 1, wherein the device is encased in a housing.

31. The device of claim 1, wherein the one or more nucleic acid molecules are immobilized directly to the capture zone membrane.

32. The device of claim 1, wherein the one or more nucleic acid molecules are immobilized indirectly to the capture zone membrane.

33. The device of claim 1, wherein the one or more nucleic acid molecules are arranged in the capture zone in the form of a dot, line, curve, band, cross or combinations thereof.
